(19) United States
(12) Patent Application Publication
(10) Pub. No.: US 2003/0199800 A1
(43) Pub. Date: Oct. 23, 2003

(54) Bandage including perforated gel

(76) Inventor: John M. Levin, New Milford, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/260,166

(22) Filed: Sep. 30, 2002

Related U.S. Application Data
(60) Provisional application No. 60/373,302, filed on Apr. 17, 2002.

(51) Int. Cl. A61F 13/00

(52) U.S. Cl. 602/43

(57) ABSTRACT

Protective bandages in accordance with this invention include an anchoring strip having a protective layer on a lower surface thereof for overlying a desired area on a person's skin. Force-transmission-impeding means is provided on the side of the anchoring strip opposed to the side including the protective layer. The force-transmission-impeding means includes a top sheet adhered adjacent peripheral edges thereof to an upper surface of the anchoring strip to define an internal compartment between the anchoring strip and the top sheet. A compressive cushioning member is provided within the internal compartment for absorbing compressive forces, and low friction, relatively movable confronting surfaces are provided within the internal compartment spaced from the upper surface of the anchoring strip to absorb shear forces imposed upon the bandage. In certain embodiments of this invention, two pairs of low friction, relatively movable confronting surfaces are provided, whereas in another embodiment of this invention only one pair of low friction, relatively movable confronting surfaces is provided.

### CROSS-REFERENCE TO RELATED APPLICATIONS

This application claims priority based on Provisional Application Serial No. 60/373,302 filed on Apr. 17, 2002 entitled BANDAGE INCLUDING PERFORATED GEL, and all of whose entire disclosures are incorporated by reference herein.

### BACKGROUND OF THE INVENTION

1. Field of the Invention

This invention relates generally to protective bandages, and more particularly, to protective bandages including a gel that holds it shape and which includes a plurality of perforations therethrough.

2. Description of Related Art

Protective bandages are used for a wide variety of applications, including the protection of skin grafts, surgical wounds, traumatic wounds, e.g., cuts and abrasions, decubitus ulcers (or prevention thereof) that most commonly form in areas of the sacral, hip, buttocks and elbows. These protective bandages come in a variety of sizes to protect injured skin areas of varying sizes. For example, bandages having a length in the range of 1″ to 2″ generally are provided to protect blisters and similar skin injuries. Bandages that are generally 3″ to 4″ in length are commonly employed to protect small surgical incisions of the type that are made in hernia operations and in other minor surgical procedures. Bandages that are 6″ to 8″ in length, and even longer, are employed to protect larger incisions that are made in connection with major surgical procedures, e.g., back surgery and heart related surgeries. Bandages employed to protect surgical incisions generally are in the range of 2″ to 3″ wide.

Common prior art protective bandages generally include an inner gauze or other protective layer or member surrounded by an adhesive layer. The gauze or other protective member is placed over the area of the skin to be protected, and is adhered in that position by the surrounding adhesive layer. While these bandages do protect the injured skin area from direct external contact with other surfaces and may provide a cushion against compression or frictional forces (e.g., U.S. Pat. Nos. 1,913,928, (Kauffman); 5,170,781 (Loomis); and 5,945,679, (Baranitsky)) while conveying fluids, e.g., sweat, wound fluids, etc., away from the wound.

In view of the deficiencies existing with prior art bandages, a need exists for protective bandages of the type that effectively protect the wound from compressive forces imposed upon the bandage while permitting the transfer of fluids, e.g., sweat, wound liquids, etc., away from the wound and surrounding skin. It is to such a bandage that the present invention relates.

### BRIEF SUMMARY OF THE INVENTION

A protective bandage for skin surfaces. The bandage comprises: a first member (e.g., a gel-type material such as ClearSite® or GAK) positioned against the skin for absorbing compressive forces applied against the bandage wherein the first member comprises a plurality of perforations therethrough to permit passage of fluid (e.g., sweat, blood, other liquids from the wound or skin) from the skin to a first surface of the first member, and wherein the first member comprises a first periphery that can be releasably-coupled to the skin surface; and a second member disposed on the first member and wherein the second member comprises an absorptive layer (e.g., gauze, sponge, etc.) having a second periphery that can be releasably coupled to the first periphery.

A protective bandage for skin surfaces. The bandage comprises: a compressive cushioning member (e.g., a gel-type material such as ClearSite® or GAK) positioned against the skin for absorbing compressive forces applied against the bandage, wherein the compressive cushioning member comprises a plurality of perforations therethrough to permit passage of fluid (e.g., sweat, blood, other liquids from the wound or skin) from the skin to a first surface of the compressive cushioning member and wherein the compressive cushioning member comprises a first periphery that can be releasably-coupled to the skin surface; an absorptive layer (e.g., gauze, sponge, etc.) disposed on the compressive cushioning member, and wherein the absorption layer comprises a second surface for absorbing the fluid; and an outer covering having a second periphery that can be coupled to the first periphery of the compressive cushioning member, and wherein the outer covering encloses the compressive cushioning member and the absorption layer therein.

A method for protecting a wound while permitting the transfer of fluid away from the wound and its surrounding skin. The method comprises the steps of: positioning a cushion member (e.g., a gel-type material such as ClearSite® or GAK) having a plurality of perforations therethrough against a wound wherein the plurality of perforations convey fluids (e.g., sweat, blood, other liquids from the wound or skin) away from the wound and surrounding skin to a first surface of the cushion member; positioning an absorptive layer (e.g., gauze, sponge, etc.) against the first surface; and covering the combination of the absorptive layer and the cushion member with a cover.

A method for protecting a wound while permitting the transfer of fluid away from the wound and its surrounding skin. The method comprises the steps of: positioning a cushion member (e.g., a gel-type material such as ClearSite® or GAK) having a plurality of perforations therethrough against a wound and wherein the plurality of perforations convey fluids (e.g., sweat, blood, other liquids from the wound or skin) away from the wound and surrounding skin to a first surface of the cushion member; and covering the cushion member with a cover having an absorptive layer (e.g., gauze, sponge, etc.) coupled to the cover.

A protective bandage for skin surfaces wherein the bandage comprises a body having a first side treated with an adhesive and includes a smaller absorptive layer on the first side and wherein the body also comprises a free-standing tab that can be seized by a user.

A method for making a protective bandage that can be manipulated regardless of its location. The method comprises the steps of: providing a body having an adhesive on a side of the body; positioning a smaller absorptive layer on the first side; and providing a free-standing tab on the body wherein the tab permits a user to manipulate the body of the bandage.

### BRIEF DESCRIPTION OF SEVERAL VIEWS OF THE DRAWINGS

The invention will be described in conjunction with the following drawings in which like reference numerals designate like elements and wherein:

FIG. 1 is an isometric view of the bandage including perforated gel of the present invention;

FIG. 2 is an exploded view of the bandage including perforated gel of FIG. 1;

FIG. 3 is a cross-sectional view of the bandage of FIG. 1 taken along line 3-3;

FIG. 4 is an isometric view of an all-purpose bandage including pull tab and showing a portion of the bandage being pulled away from its release liner; and

FIG. 5 is a partial cross-sectional view of the all-purpose bandage taken along line 5-5 of FIG. 4.

### DETAILED DESCRIPTION OF THE INVENTION

The present invention 20 comprises a protective bandage having some similarities to the bandages disclosed in Application Ser. No. 60/349,587 filed on Jan. 18, 2002 entitled COMPARTMENTALIZED BANDAGE and in Application Ser. No. 09/615,211 filed on Jul. 13, 2000 entitled PROTECTIVE BANDAGES INCLUDING FORCE-TRANSMISSION-IMPEDING MEMBERS THEREIN, both of whose entire disclosures are incorporated by reference herein. However, unlike those bandages, the present invention 20 forms a simpler construction.

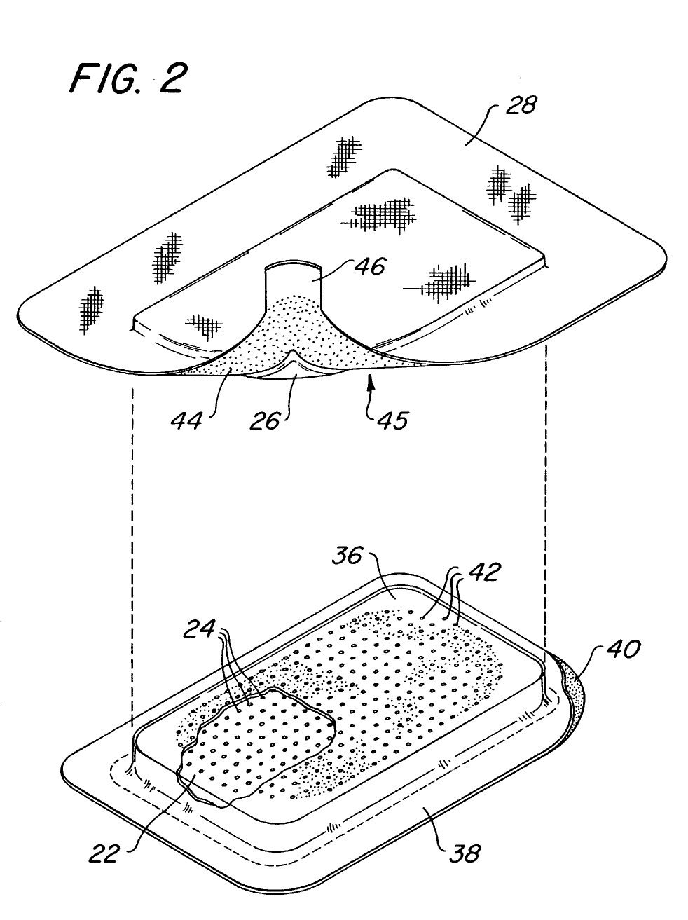

In particular, the present invention 20 basically comprises a compressive cushioning layer 22, e.g., a gel-type material, having perforations 24 (FIG. 2), an absorptive layer 26 and an outer covering 28.

The compressive cushioning layer 22 absorbs compressive forces applied against the bandage 20 when it is in place over a wound. The compressive cushioning layer 22 preferably comprises a gel-type material 22 which essentially maintains its shape unless directly exposed to external forces and comprises a plurality of perforations 24 that pass through the entire thickness of the gel-type material 22 to allow the absorption layer 26 to absorb fluid from a wound 30 against which the gel-type material 22 is positioned. For example, sweat, blood, water, or any other liquid that forms on or near the wound 30, including from surrounding skin 32, can migrate away from the wound30/skin 32 and through the perforations 24 towards a surface 25 (FIG. 3). As shown in FIG. 3, a bottom surface 34 of the gel-type material 22 is positioned directly on a wound 30. The importance of providing a means for fluid to migrate away from the wound 30 is because a wet wound in almost all cases is predisposed to infection and skin maceration. Without the perforations 24 fluid collects under the gel-type material 22. Preferably, the gel-type material 22 is clear (e.g., ClearSite® by Conmed Corporation of Utica, N.Y. or other transparent synthetic gel material, such as the gel-type material sold under the name “GAK”) for wound examination. Furthermore, the gel-type material 22 is covered by an outer, low friction wrap of plastic film 36 (FIG. 2) whose periphery 38 includes an adhesive surface 40 (e.g., a biocompatible adhesive) that is applied to the skin 32; the plastic film 36 also comprises a plurality of perforations 42 to allow passage of fluid into the absorption layer 26. Thus, the bandage 20 is releasably secured against the wound 30 via this adhesive surface 40. The bottom surface 34 of the gel-type material 22 is in direct contact with the wound 30. The wound 30 could even be treated with H₂O₂ and provodine-iodine (Betadine) to suppress growth of microbes. The perforations 24 and 42 are a very important aspect of the present invention 20 since the use of the gel-type material 22 directly on the skin 32 or wound 30 without the perforations 24/42 accumulates sweat and wound fluids beneath the gel-type material 22 which is undesirable.

The absorptive layer 26 is positioned on top of the film 36 and may comprise any type of absorptive material, such as gauze or a sponge, etc. Thus, fluids from the wound 30 are able to escape the wound 30 by passing through the plurality of apertures 24 in the gel-type material 22, through the plurality of apertures 42 in the film 36 and then into the absorptive layer 26.

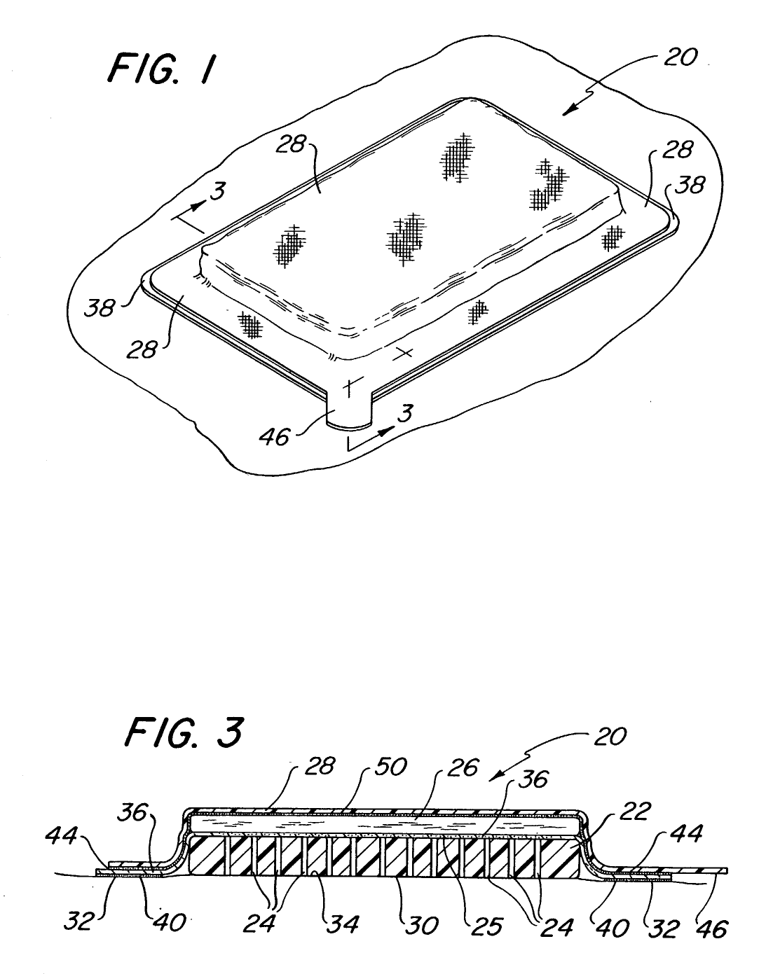

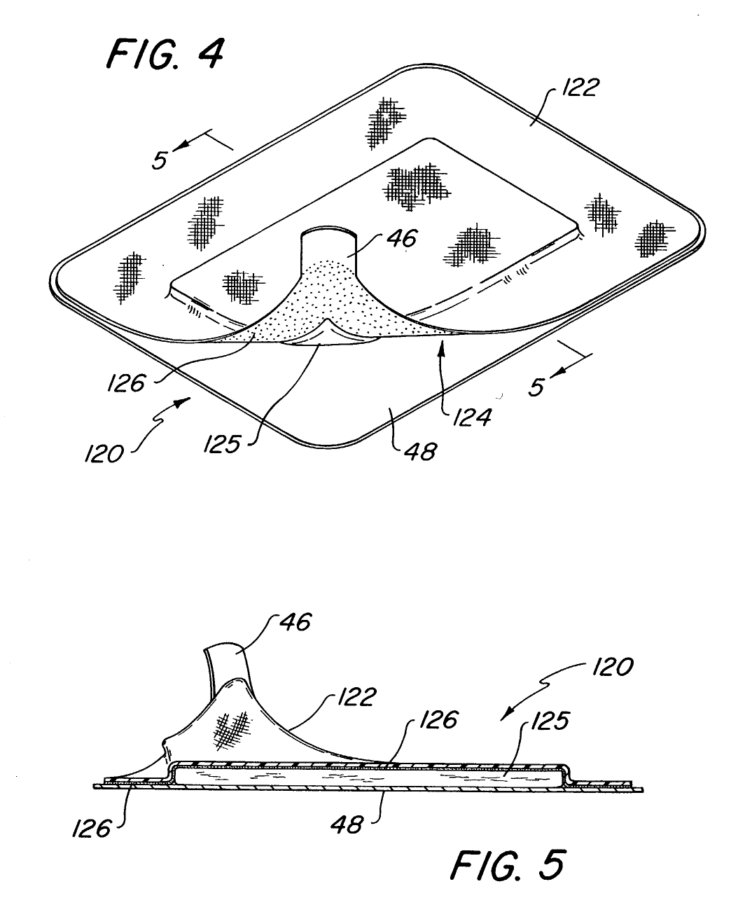

The outer covering 28 comprises an adhesive 44 on its underside surface 45 which corresponds to the periphery 38 of the plastic film 36. The outer covering 28 is, thus, coupled to the periphery 38 of the film 36 and not to the skin 32 of the living being, as shown most clearly in FIGS. 1 and 3. With the outer covering 28 installed as shown in FIGS. 1 and 3, the bandage 20 is complete. The outer covering 28 also comprises a tab 46 that can be seized by the user (e.g., attendant or patient, etc.) in handling the outer covering 28. For example, the tab 46 is free, i.e., it does comprise any adhesive and can be easily grabbed by the user to remove the covering 28 from its corresponding release sheet 48 (FIG. 4); similarly, when the wound 30 needs to be inspected and/or to change out the absorptive layer 26, the outer covering 28 can be easily removed because the user can seize the free tab 46 and pull the outer covering 28 away from the periphery 38 of the film 36.

It should be understood that the adhesive property of the adhesive 44 on the underside surface 45 is similar in strength to the adhesive used in memo papers sold under the name Post-It® (sold by 3M Corporation). If, on the other hand, a stronger adhesive were used, the user could inadvertently pull the plastic film 38 and the gel-type material 22 off of the skin 32 and wound 30 when attempting to only remove the covering 28 in order to change out the absorptive layer 26.

It should also be understood that it is within the broadest scope of the present invention to have the absorptive layer 26 be either coupled to the inside surface of the outer covering 28 using a coupling means (e.g., an adhesive 50, FIG. 3) or to be a free element that is disposed on the gel-type material 22 which is then covered by the outer covering 28.

It should also be understood that although it is preferable to use a transparent gel-type material 22 (e.g., to facilitate viewing the wound 30 without the need to remove the gel-type material 22), it is within the broadest scope of this invention to include an opaque gel-type material 22. The important feature of the gel-type material 22, transparent or opaque, is the inclusion of the perforations 24 as discussed previously.

Using the protective bandage 20 of the present invention, the unique design allows one to change the absorptive layer 26 without touching, abrading or exposing the skin 32.

FIGS. 4-5 disclose an all-purpose bandage 120 that includes the pull tab 46. In particular, the bandage 120 is similar to conventional bandages in that it comprises a body 122 having an underside surface 124 treated with an adhesive 126 (e.g., a biocompatible adhesive) and includes a smaller-sized absorptive layer 125 coupled to the underside surface 124. However, unlike conventional bandages, the bandage 120 comprises the pull tab 46 that provides the user with something to hold when removing the liner 48. But more importantly, because the tab 46 is free and not adhesively coupled to the skin, when it is necessary to remove the bandage 120 from the skin, the user can easily remove the bandage 120 by seizing the pull tab 46 and pulling the bandage 120 off the skin (not shown). In contrast, when attempting to remove a conventional bandage from the skin, the user needs to pry an edge of the bandage off from the skin which is not easily accomplished due to the nature of the adhesive.

While the invention has been described in detail and with reference to specific examples thereof, it will be apparent to one skilled in the art that various changes and modifications can be made therein without departing from the spirit and scope thereof.

What is claimed is:

1. A protective bandage for skin surfaces, said bandage comprising:
a first member positioned against the skin for absorbing compressive forces applied against the bandage, said first member comprising a plurality of perforations therethrough to permit passage of fluid from the skin to a first surface of said first member, said first member comprising a first periphery that can be releasably-coupled to the skin surface; and
a second member disposed on said first member and wherein said second member comprises an absorptive layer having a second periphery that can be releasably coupled to said first periphery.

2. The protective bandage of claim 1 wherein said first member comprises a gel-type material.

3. The protective bandage of claim 2 wherein said gel-type material is a non-flowing gel-type material.

4. The protective bandage of claim 2 wherein said gel-type material is transparent.

5. The protective bandage of claim 1 wherein said absorptive layer comprises gauze.

6. The protective bandage of claim 1 wherein said second member further comprises a tab that can be seized by a user.

7. The protective bandage of claim 3 wherein gel-type material is covered by a plastic film having perforations and wherein said plastic film comprises said second periphery.

8. A protective bandage for skin surfaces, said bandage comprising:
a compressive cushioning member positioned against the skin for absorbing compressive forces applied against the bandage, said compressive cushioning member comprising a plurality of perforations therethrough to permit passage of fluid from the skin to a first surface of said compressive cushioning member, said compressive cushioning member comprising a first periphery that can be releasably-coupled to the skin surface;
an absorptive layer disposed on said compressive cushioning member, said absorption layer comprising a second surface for absorbing said fluid; and
an outer covering having a second periphery that can be coupled to said first periphery of said compressive cushioning member, said outer covering enclosing said compressive cushioning member and said absorption layer therein.

9. The protective bandage of claim 8 wherein said compressive cushioning member includes a gel-type material.

10. The protective bandage of claim 9 wherein said gel-type material is a non-flowing gel-type material.

11. The protective bandage of claim 9 wherein said gel-type material is transparent.

12. The protective bandage of claim 8 wherein said absorption layer comprises gauze.

13. The protective bandage of claim 8 wherein said second member further comprises a tab that can be seized by a user.

14. The protective bandage of claim 10 wherein gel-type material is covered by a plastic film having perforations and wherein said plastic film comprises said second periphery.

15. A method for protecting a wound while permitting the transfer of fluid away from the wound and its surrounding skin, said method comprising the steps of:
positioning a cushion member having a plurality of perforations therethrough against a wound, said plurality of perforations conveying fluids away from the wound and surrounding skin to a first surface of said cushion member;
positioning an absorptive layer against said first surface; and
covering said combination of said absorptive layer and said cushion member with a cover.

16. The method of claim 15 wherein said step of positioning a cushion member comprises adhesively coupling said cushion member to the skin surrounding the wound along a first periphery of said cushion member.

17. The method of claim 16 wherein said step of covering said combination of said absorptive layer and said cushion member comprises adhesively coupling a second periphery of said cover to said first periphery of said cushion member.

18. A method for protecting a wound while permitting the transfer of fluid away from the wound and its surrounding skin, said method comprising the steps of:
positioning a cushion member having a plurality of perforations therethrough against a wound, said plurality of perforations conveying fluids away from the wound and surrounding skin to a first surface of said cushion member; and
covering said cushion member with a cover having an absorptive layer coupled to the cover.

19. The method of claim 18 wherein said step of positioning a cushion member comprises adhesively coupling said cushion member to the skin surrounding the wound along a first periphery of said cushion member.

20. The method of claim 18 wherein said step of covering said cushion member comprises adhesively coupling a second periphery of said cover to said first periphery of said cushion member.

21. A protective bandage for skin surfaces, said bandage comprising a body having a first side treated with an adhesive and including a smaller absorptive layer on said first side and wherein said body comprises a free-standing tab that can be seized by a user.

22. The protective bandage of claim 21 further comprising a release liner releasably coupled to said first side.

23. A method for making a protective bandage that can be manipulated regardless of its location, said method comprises the steps of:
providing a body having an adhesive on a side of said body;
positioning a smaller absorptive layer on said first side; and
providing a free-standing tab on said body, said tab permitting a user to manipulate the body of said bandage.
